(19) United States
(12) Patent Application Publication
(10) Pub. No.: US 2026/0287495 A1
(43) Pub. Date: Sep. 24, 2026

(54) CORROSION REPRODUCING DEVICE, CONTROL DEVICE, EVALUATION SYSTEM, CONTROL METHOD, AND PROGRAM

(71) Applicant: NTT, Inc., Tokyo (JP)

(72) Inventors: Yosuke TAKEUCHI, Tokyo (JP); Yasuhiro MATSUMOTO, Tokyo (JP); Toshimitsu IZUMI, Tokyo (JP)

(73) Assignee: NTT, Inc., Tokyo (JP)

(21) Appl. No.: 18/993,837

(22) PCT Filed: Jul. 15, 2022
(86) PCT No.: PCT/JP2022/027943

§ 371 Date: Jan. 13, 2025

(51) Int. Cl. G01N 17/04; G05B 19/402

(52) U.S. Cl. CPC G01N 17/04; G05B 19/402

(57) ABSTRACT

A corrosion reproduction device (1) according to the present disclosure includes a container (111), a calcium hydroxide aqueous solution (112) accommodated in the container (111), and a reinforcing bar member (113) immersed in the calcium hydroxide aqueous solution (112).

### TECHNICAL FIELD

The present disclosure relates to a corrosion reproduction device, a control device, an evaluation system, a control method, and a program.

### BACKGROUND ART

It is known that reinforced concrete changes over time. Specifically, as illustrated in the upper part of FIG. 7, neutralization of a portion adjacent to the atmosphere in concrete 91 constituting reinforced concrete proceeds. As a result, corrosion of a reinforcing bar 92 provided in the concrete 91 constituting the reinforced concrete progresses. A corrosion product (for example, rust) 93 of the reinforcing bar 92 then expands. In the example illustrated in FIG. 7, a portion surrounded by a broken line is an expanded portion 93a of the corrosion product. Then, the expansion of the corrosion product causes a crack in the concrete. As a result, a part of the concrete (the concrete portion 91a illustrated in the upper part of FIG. 7) in contact with the expanded portion 93a is peeled off as illustrated in the middle part of FIG. 7, and the corrosion of the reinforcing bar 92 further proceeds. As described above, when a part of the concrete is peeled off, a part of the reinforcing bar 92 is further exposed, and the exposed portion (exposed reinforcement portion) corrodes, so that durability of reinforced concrete decreases.

Further, as the corrosion of the exposed reinforcement portion progresses, the corrosion product 93 further expands to the portion 93b illustrated in the middle part of FIG. 7, and a further part of the concrete (the concrete portion 91b illustrated in the middle part of FIG. 7) peels off as illustrated in the lower part of FIG. 7, so that the exposed reinforcement portion expands. As described above, due to repeated expansion of the corrosion product, peeling of concrete, and corrosion of the exposed reinforcement portion, the durability of reinforced concrete is continuously reduced.

Conventionally, the temporal change of the reinforcing bar, which causes a decrease in durability of reinforced concrete as described above, has been evaluated by investigating an actual structure. In addition, as described in Non Patent Literature 1, it is also known that evaluation is performed by performing a corrosion acceleration test on a model specimen. Furthermore, Non Patent Literature 2 discloses that phenolphthalein is used in a corrosion acceleration test, and Non Patent Literature 3 discloses that a corrosion rate of a reinforcing bar is measured and a reinforcing bar corrosion situation is evaluated by a natural potential method.

### CITATION LIST

### Non Patent Literature

Non Patent Literature 1: “TIME-DEPENDENT CORROSION BEHAVIOR OF STRUCTURAL STEEL MEMBERS IN BOUNDARY WITH CONCRETE” by Shigenobu Kainuma and three others, Japanese Journal of JSCE vol. 70, pp. 97-114 January 2005
Non Patent Literature 2: Lukuan Q I two others, “STUDY ON CORROSION OF REINFORCING BAR DUE TO CONCRETE NEUTRALIZATION UNDER ALTERNATE DRYING AND WETTING CONDITIONS”, Japanese Journal of JSCE vol. 54, pp. 1-11 February 2002
Non Patent Literature 3: “Deterioration Prediction of Concrete Structures Based on the Exposure Test Results Concerning Cracking and Rebar Corrosion”, Seiichi Tottori and two others, Concrete Research and Technology, Vol. 10, No. 3. pp. 1-15 September 1999

### SUMMARY OF INVENTION

### Technical Problem

However, in order to investigate an actual structure of reinforced concrete, it is necessary to secure an object to be investigated, and in order to perform a corrosion acceleration test on a specimen of reinforced concrete, it is necessary to prepare a specimen. As described above, since the preparation requires time and cost, there is a problem that it is difficult to evaluate reinforced concrete.

An object of the present disclosure made in view of such circumstances is to provide a corrosion reproduction device, a control device, an evaluation system, a control method, and a program capable of easily reproducing corrosion of reinforced concrete.

### Solution to Problem

In order to solve the above problem, a corrosion reproduction device according to the present disclosure includes a container, a calcium hydroxide aqueous solution accommodated in the container, and a reinforcing bar member immersed in the calcium hydroxide aqueous solution.

In order to solve the above problem, a control device according to the present disclosure is a control device that controls a corrosion reproduction device including a container, a calcium hydroxide aqueous solution accommodated in the container, and a reinforcing bar member immersed in the calcium hydroxide aqueous solution, the control device including: a corrosion amount detection unit that detects a corrosion amount of a corrosion product generated in the reinforcing bar member; a relative position determination unit that determines a relative position of the reinforcing bar member with respect to an interface of the calcium hydroxide aqueous solution on the basis of the corrosion amount; and a relative position control unit that performs control such that the reinforcing bar member is disposed at the relative position with respect to the interface.

In order to solve the above problem, an evaluation system according to the present disclosure is an evaluation system including a corrosion reproduction device and a control device, the corrosion reproduction device including a container, a calcium hydroxide aqueous solution accommodated in the container, and a reinforcing bar member immersed in the calcium hydroxide aqueous solution, the control device including: a corrosion amount detection unit that detects a corrosion amount of a corrosion product generated in the reinforcing bar member; a relative position determination unit that determines a relative position of the reinforcing bar member with respect to an interface of the calcium hydroxide aqueous solution on the basis of the corrosion amount; and a relative position control unit that performs control such that the reinforcing bar member is disposed at the relative position with respect to the interface.

In order to solve the above problem, a control method according to the present disclosure is a control method performed by a control device that evaluates a reinforcing bar member in a corrosion reproduction device including a container, a calcium hydroxide aqueous solution accommodated in the container, and the reinforcing bar member immersed in the calcium hydroxide aqueous solution, the control method including steps of: detecting a corrosion amount of a corrosion product generated in the reinforcing bar member; determining a relative position of the reinforcing bar member with respect to an interface of the calcium hydroxide aqueous solution on the basis of the corrosion amount; and performing control such that the reinforcing bar member is disposed at the relative position with respect to the interface.

Furthermore, in order to solve the above problem, a program according to the present disclosure causes a computer to operate as the control device described above.

### Advantageous Effects of Invention

According to a corrosion reproduction device, a control device, an evaluation system, a control method, and a program according to the present disclosure, it is possible to easily reproduce corrosion of reinforced concrete.

### BRIEF DESCRIPTION OF DRAWINGS

FIG. 1 is a schematic diagram illustrating an example of an evaluation system according to the present embodiment of the present disclosure.

FIG. 2 is a schematic diagram illustrating an example of a corrosion reproduction device and a measurement unit illustrated in FIG. 1.

FIG. 3A is a schematic diagram illustrating an example of a reproduction unit illustrated in FIG. 2.

FIG. 3B is a schematic diagram illustrating an example in which the example of the reproduction unit illustrated in FIG. 3A is changed.

FIG. 3C is a schematic diagram illustrating an example in which the example of the reproduction unit illustrated in FIG. 3B is further changed.

FIG. 4 is a diagram illustrating an example of a corrosion rate of a reinforcing bar measured by the measurement unit illustrated in FIG. 1.

FIG. 5 is a flowchart illustrating an example of operation using the control device illustrated in FIG. 1.

FIG. 6 is a diagram illustrating an example of a hardware configuration of the control device illustrated in FIG. 1.

FIG. 7 is a schematic diagram for explaining corrosion in an actual structure of reinforced concrete.

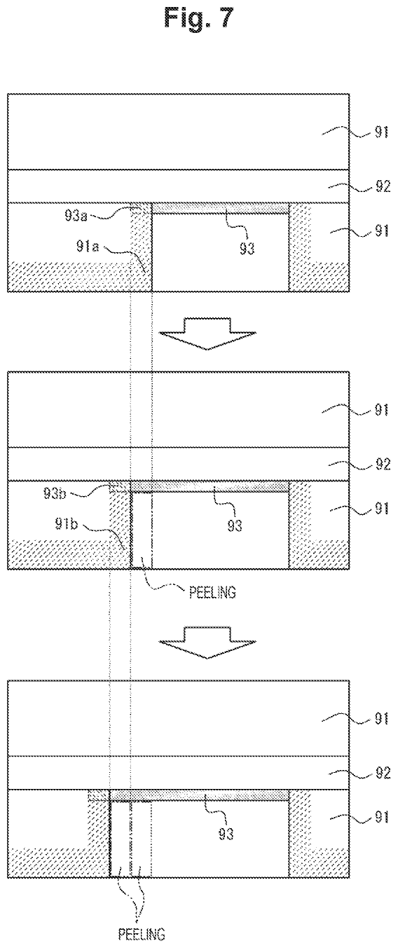

### DESCRIPTION OF EMBODIMENTS

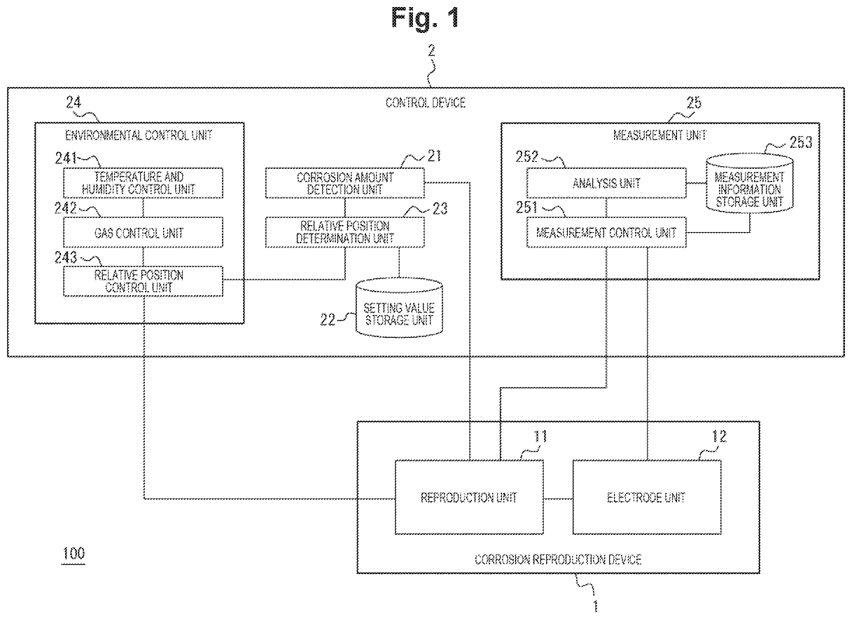

An overall configuration of the present embodiment will be described with reference to FIG. 1. FIG. 1 is a schematic diagram illustrating an example of an evaluation system 100 according to the present embodiment. The evaluation system 100 includes a corrosion reproduction device 1 and a control device 2.

The corrosion reproduction device 1 includes a reproduction unit 11 and an electrode unit 12. Note that the corrosion reproduction device 1 does not need to include the electrode unit 12.

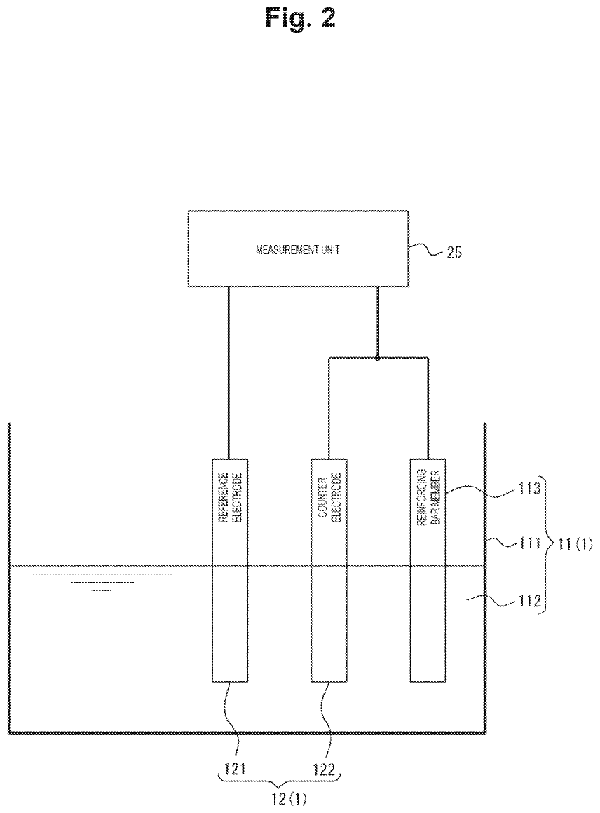

As illustrated in FIG. 2, the reproduction unit 11 includes a container 111, a calcium hydroxide aqueous solution 112, and a reinforcing bar member 113.

The container 111 is a housing that can accommodate liquid. In the present embodiment, the container 111 accommodates the calcium hydroxide aqueous solution 112.

The calcium hydroxide aqueous solution 112 is a liquid in which calcium hydroxide is dissolved in water. Calcium hydroxide is neutralized by being combined with carbon dioxide in the air and forming calcium carbonate. The calcium hydroxide aqueous solution 112 is accommodated in the container 111.

Phenolphthalein may be injected into the calcium hydroxide aqueous solution 112. As a result, the neutralized portion in the calcium hydroxide aqueous solution 112 is colored, and it is easy to distinguish the neutralized portion in the calcium hydroxide aqueous solution 112.

The calcium hydroxide aqueous solution 112 may be stirred when a portion from an interface which is a boundary with the atmosphere to a desired depth is neutralized. The calcium hydroxide aqueous solution 112 may be stirred using, for example, a magnetic stirrer or the like. As a result, a neutralized state of the calcium hydroxide aqueous solution 112 from the water surface to a desired depth is maintained, and the reinforced concrete in this state can be evaluated.

The calcium hydroxide aqueous solution 112 may be gelled. For example, the calcium hydroxide aqueous solution 112 to which a gelling agent such as gelatin or agar is added is heated. Then, the reinforcing bar member 113 is immersed in the heated calcium hydroxide aqueous solution 112, and the calcium hydroxide aqueous solution 112 is cooled and gelled. As a result, it is possible to suppress the neutralization of the calcium hydroxide aqueous solution 112 from becoming non-uniform due to the convection of the calcium hydroxide aqueous solution 112. In addition, it is possible to suppress a change in the water level due to evaporation of the calcium hydroxide aqueous solution 112. Therefore, the corrosion situation in the corrosion reproduction device 1 can be stably measured.

The reinforcing bar member 113 is immersed in the calcium hydroxide aqueous solution 112 accommodated in the container 111. The reinforcing bar member 113 may be immersed in the calcium hydroxide aqueous solution 112 such that a part in the vertical direction is exposed over the entire circumferential direction. As a result, it is possible to reproduce in a pseudo manner reinforced concrete in which a part in the normal direction of the interface of the reinforcing bar is exposed over the entire circumferential direction. The reinforcing bar member 113 may be immersed in the calcium hydroxide aqueous solution 112 such that a part in the circumferential direction of a part of the interface in the normal direction is exposed to the atmosphere and the other part in the circumferential direction is covered with an epoxy resin or the like. In this case, reinforced concrete in which a part in the circumferential direction of a part in the normal direction of the interface of the reinforcing bar member 113 is exposed can be reproduced in a pseudo manner. As described above, the reinforcing bar member 113 is immersed in accordance with the state of the actual structure of reinforced concrete to be evaluated, which is reproduced in a pseudo manner by the reinforcing bar member 113.

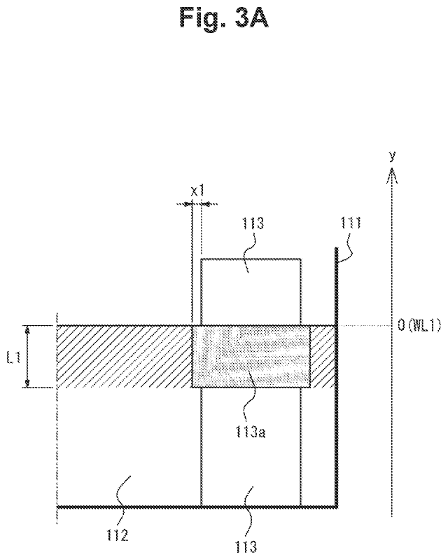

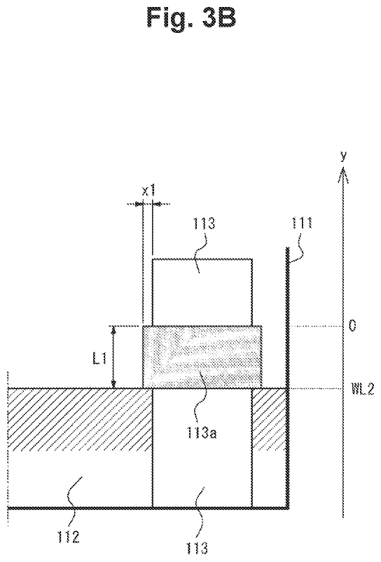

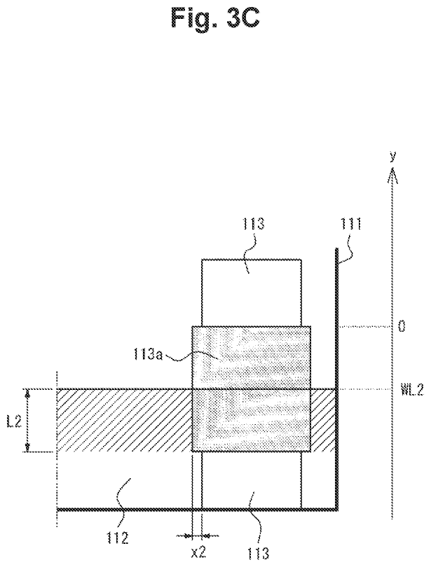

By configuring the reproduction unit 11 as described above, as illustrated in FIG. 3A, the vicinity of the interface of the calcium hydroxide aqueous solution 112 (hatched portion in FIG. 3A) is neutralized by carbon dioxide in the atmosphere. This reproduces in a pseudo manner that the vicinity of the surface of concrete is neutralized by carbon dioxide in the atmosphere in the actual structure of reinforced concrete.

Then, in the reproduction unit 11, a portion of the reinforcing bar member 113 in the vicinity of the neutralized portion of the calcium hydroxide aqueous solution 112 corrodes to generate a corrosion product 113a and expands. This reproduces in a pseudo manner that, in the actual structure of reinforced concrete, a portion of the reinforcing bar in the vicinity of the neutralized portion of the concrete corrodes to generate a corrosion product, and expands. When the reinforcing bar member 113 is a rod-shaped member extending in the normal direction of the interface, a corrosion width X is the radial length of the corrosion product 113a.

In the reproduction unit 11, as illustrated in FIG. 3B, the portion on the interface side of the calcium hydroxide aqueous solution 112 is drained from the state illustrated in FIG. 3A to reduce the water level, so that the concrete peeling is reproduced in a pseudo manner.

When the portion on the interface side of the calcium hydroxide aqueous solution 112 is drained and the water level decreases, as illustrated in FIG. 3C, the portion of the reinforcing bar member 113 in the vicinity of the neutralized portion of the calcium hydroxide aqueous solution corrodes again, and a corrosion product 113a is generated and expands. As described above, by repeating the decrease in the water level, the neutralization of the calcium hydroxide aqueous solution, and the expansion of the corrosion product 113a, the peeling of the concrete, the neutralization of the concrete, and the repeated corrosion of the reinforcing bar in the actual structure of reinforced concrete are reproduced.

As illustrated in FIG. 2, the electrode unit 12 includes a reference electrode 121 and a counter electrode 122.

The reference electrode 121 is an electrode that provides a reference potential at the time of measuring the potential.

The counter electrode 122 is one of a pair of electrodes when the reinforcing bar member 113 is used as a working electrode.

Returning to FIG. 1, the control device 2 controls the corrosion reproduction device 1. The control device 2 includes a corrosion amount detection unit 21, a setting value storage unit 22, a relative position determination unit 23, an environmental control unit 24, and a measurement unit 25. The corrosion amount detection unit 21, the relative position determination unit 23, and the environmental control unit 24 include a controller. The controller may be configured by dedicated hardware such as an application specific integrated circuit (ASIC) or a field-programmable gate array (FPGA), may be configured by a processor, or may be configured to include both dedicated hardware and a processor. The setting value storage unit 22 includes a memory. The memory may include a hard disk drive (HDD), a solid state drive (SSD), an electrically erasable programmable read-only memory (EEPROM), a read-only memory (ROM), and a random access memory (RAM). The measurement unit 25 includes a memory and a controller. Note that the control device 2 does not need to include the setting value storage unit 22 and the measurement unit 25.

The corrosion amount detection unit 21 detects the corrosion amount of the corrosion product 113a generated in the reinforcing bar member 113 accommodated in the calcium hydroxide aqueous solution 112. The corrosion amount includes a corrosion width X and a corrosion length L. The corrosion width X is an expansion amount of the corrosion product 113a in a direction parallel to the interface of the calcium hydroxide aqueous solution 112. In the examples of FIGS. 3A and 3B, the corrosion width X is “X1”, and in the example of FIG. 3C, the corrosion width X is “X2”. The corrosion length L is a length in the normal direction of the interface of the corrosion product 113a generated in the portion of the reinforcing bar member 113 immersed in the calcium hydroxide aqueous solution 112. In the example of FIG. 3A, the corrosion length L is “L1”, and in the example of FIG. 3C, the corrosion length L is “L2”.

The corrosion amount detection unit 21 can detect the corrosion amount by any method.

As a first example, the corrosion amount detection unit 21 may detect a cracked corrosion amount β on the basis of a compressive strength σ (N/mm²) and a covering thickness c (mm). Specifically, the corrosion amount detection unit 21 can detect the cracked corrosion amount β (mg/cm²) using Expression (1) described in Non Patent Literature 4 below. The compressive strength σ and the covering thickness c are values determined by specifications of a structure including reinforced concrete reproduced in a pseudo manner by the corrosion reproduction device 1 of the present embodiment, and are stored in advance in a memory included in the control device 2. The corrosion amount detection unit 21 may detect the above-described corrosion amount on the basis of the cracked corrosion amount β.
Non Patent Literature 4: Yasuji Shinohara and three others, “CORROSION BEHAVIORS BEFORE AND AFTER CRACKING INDUCED BY CORRODING BARS AND CRACK PROPAGATIONS”, J. Struct. Constr. Eng., AIJ., Vol: 81, No. 728, pp. 1609-1618 October 2016

[Math. 1]

β=(−0.13σ²+11.5σ−160)(e^{0.01e}−1) (1)

As a second example, the corrosion amount detection unit 21 may detect the corrosion amount on the basis of an image generated by imaging the reinforcing bar member 113 by an imaging device such as a video camera. For example, the corrosion amount detection unit 21 may extract a portion having features of the image of the corrosion product 113a from the image generated by the imaging device, and detect the corrosion width X and the corrosion length L on the basis of the width and the length of the portion, respectively.

As a third example, the corrosion amount detection unit 21 may detect the above-described corrosion amount on the basis of the weight of the reinforcing bar member 113 measured by a weight measurement mechanism. The weight measurement mechanism may be provided in a grip portion or the like of the reinforcing bar member 113. For example, the corrosion amount detection unit 21 may measure the weight of the corrosion product 113a increased from a first timing to a second timing on the basis of a value obtained by subtracting the weight of the reinforcing bar member 113 measured by the weight measurement mechanism at the first timing from the weight of the reinforcing bar member 113 measured by the weight measurement mechanism at the second timing after the first timing, and detect the corrosion amount on the basis of the weight.

The setting value storage unit 22 stores a peeling corrosion width. A that is a threshold of the corrosion width X used by the relative position determination unit 23 to determine the relative position. The peeling corrosion width A is a value determined in advance by the user of the evaluation system 100 of the present embodiment, and can be, for example, a corrosion width in which peeling of concrete is expected to occur. The setting value storage unit 22 may store the peeling corrosion width A input by a user's operation, or may store the peeling corrosion width A received from another device via a communication network.

The setting value storage unit 22 stores the number of times of peeling N indicating the upper limit of the number of times the relative position determination unit 23 determines the relative position. The number of times of peeling N is a value determined in advance by the user of the evaluation system 100 of the present embodiment. The setting value storage unit 22 may store the number of times of peeling N input by a user's operation, or may store the number of times of peeling N received from another device via a communication network.

The relative position determination unit 23 determines the relative position of the reinforcing bar member 113 with respect to the interface of the calcium hydroxide aqueous solution 112 on the basis of the corrosion amount.

Specifically, the relative position determination unit 23 determines whether the corrosion width X is equal to or larger than the peeling corrosion width A. When it is determined that the corrosion width X is equal to or larger than the peeling corrosion width A, the relative position determination unit 23 measures the corrosion length L. Then, the relative position determination unit 23 determines the relative position on the basis of the corrosion length L. When it is determined that the corrosion width X is smaller than the peeling corrosion width A, the relative position determination unit 23 measures the corrosion width X again after a predetermined time, for example.

For example, the relative position determination unit 23 determines the relative position with respect to the interface of the reinforcing bar member 113 such that the interface is positioned to be a boundary between a corroded portion and a non-corroded portion of the reinforcing bar member 113 in the calcium hydroxide aqueous solution 112. In the example illustrated in FIGS. 3A and 3B, the relative position determination unit 23 determines the relative position such that the interface is relatively low with respect to the reinforcing bar member 113 by the corrosion length L1.

As will be described in detail later, the environmental control unit 24 controls the relative position of the reinforcing bar member 113 with respect to the interface to be the relative position determined by the relative position determination unit 23. When the relative position is controlled, the relative position determination unit 23 again determines whether the corrosion width X is equal to or larger than the peeling corrosion width A, and determines the relative position when it is determined that the corrosion width X is equal to or larger than the peeling corrosion width A. As a result, the position of the reinforcing bar member 113 is controlled again by the environmental control unit 24. As described above, the relative position determination unit 23 repeats the above-described processing until the number of times of determining the relative position reaches the number of times of peeling N.

The environmental control unit 24 controls the environment in which the reproduction unit 11 is provided. As illustrated in FIG. 1, the environmental control unit 24 includes a temperature and humidity control unit 241, a gas control unit 242, and a relative position control unit 243; The environmental control unit 24 can include any one or more of the temperature and humidity control unit 241, the gas control unit 242, and the relative position control unit 243.

The temperature and humidity control unit 241 controls the temperature or humidity of the environment in which the calcium hydroxide aqueous solution 112 and the reinforcing bar member 113 are provided. Specifically, the temperature and humidity control unit 241 controls a temperature adjuster such as a heater so that the temperature of the environment becomes the temperature input by the user's operation. In addition, the temperature and humidity control unit 241 controls a humidity adjuster so that the humidity of the environment becomes the humidity input by the user's operation. The temperature and humidity input by the user's operation can be set to the temperature and humidity of the environment in which the actual structure of reinforced concrete is provided, that is reproduced in a pseudo manner by the corrosion reproduction device 1 of the present embodiment.

The gas control unit 242 controls the concentration of the gas contained in the environment in which the calcium hydroxide aqueous solution 112 and the reinforcing bar member 113 are provided. The gas may be a gas in which a correlation between the concentration of the gas and neutralization of the calcium hydroxide aqueous solution 112 is clear, and can be, for example, carbon dioxide. The gas may be a gas in which a correlation between the concentration of the gas and corrosion of the reinforcing bar is clear, and may be, for example, oxygen.

Specifically, the gas control unit 242 can control the concentration of carbon dioxide so that the rate of neutralization of the calcium hydroxide aqueous solution 112 becomes the rate input by the user's operation. The gas control unit 242 can control the concentration of oxygen so that the corrosion rate of the reinforcing bar member 113 becomes the rate input by the user's operation.

The relative position control unit 243 controls the reinforcing bar member 113 to be disposed at the relative position determined by the relative position determination unit 23 with respect to the interface of the calcium hydroxide aqueous solution 112.

As an example, the relative position control unit 243 reduces the water level of the calcium hydroxide aqueous solution 112 such that the reinforcing bar member 113 is disposed at the relative position determined by the relative position determination unit 23 with respect to the interface. Specifically, as illustrated in FIGS. 3A and 3B, the relative position control unit 243 decreases the water level of the calcium hydroxide aqueous solution 112 by the corrosion length L. At this time, the relative position control unit 243 discharges the portion on the interface side (the upper portion in the example of FIGS. 3A to 3C) in the calcium hydroxide aqueous solution 112 accommodated in the container 111 from the container 111. The relative position control unit 243 can discharge the portion of the calcium hydroxide aqueous solution 112 on the interface side from the container 111 by any method. For example, the relative position control unit 243 may control the pump to suck up a portion on the interface side of the calcium hydroxide aqueous solution 112 via a drainage pipe. As a result, the relative position determination unit 23 can reduce the water level of the calcium hydroxide aqueous solution 112 so that the boundary between the corroded portion and the non-corroded portion in the reinforcing bar member 113 becomes the interface of the calcium hydroxide aqueous solution 112.

As another example, the relative position control unit 243 pulls a part of the reinforcing bar member 113 out of the calcium hydroxide aqueous solution 112 such that the reinforcing bar member 113 is disposed at the relative position determined by the relative position determination unit 23 with respect to the interface. Specifically, the relative position control unit 243 pulls out the reinforcing bar member 113 from the calcium hydroxide aqueous solution 112 so that the reinforcing bar member 113 moves by the corrosion length L. As a result, the relative position determination unit 23 can pull out a part of the reinforcing bar member 113 from the calcium hydroxide aqueous solution 112 such that the boundary between the corroded portion and the non-corroded portion in the reinforcing bar member 113 becomes the interface of the calcium hydroxide aqueous solution 112.

As described above, in the configuration in which the calcium hydroxide aqueous solution 112 is gelled, the relative position control unit 243 controls the reinforcing bar member 113 to be disposed at the relative position determined by the relative position determination unit 23 with respect to the interface by pulling out a part of the reinforcing bar member 113 from the calcium hydroxide aqueous solution 112 without decreasing the water level of the calcium hydroxide aqueous solution 112.

The measurement unit 25 includes a measurement control unit 251, an analysis unit 252, and a measurement information storage unit 253. The measurement control unit 251 and the analysis unit 252 include a controller. The measurement information storage unit 253 includes a memory.

The measurement control unit 251 measures the corrosion state of the reinforcing bar member 113 by applying an electrical signal to the reference electrode 121, the counter electrode 122, and the reinforcing bar member 113 as a working electrode using a natural potential method, a polarization resistance method, or the like. For example, the measurement control unit 251 may measure the corrosion state on the basis of the current measured by applying a voltage across the reference electrode 121, and the counter electrode 122 and the reinforcing bar member 113. The measurement control unit 251 may measure the corrosion state of the basis of the voltage between the reference electrode 121, and the counter electrode 122 and the reinforcing bar member 113 measured by applying a current to the reference electrode 121, and the counter electrode 122 and the reinforcing bar member 113.

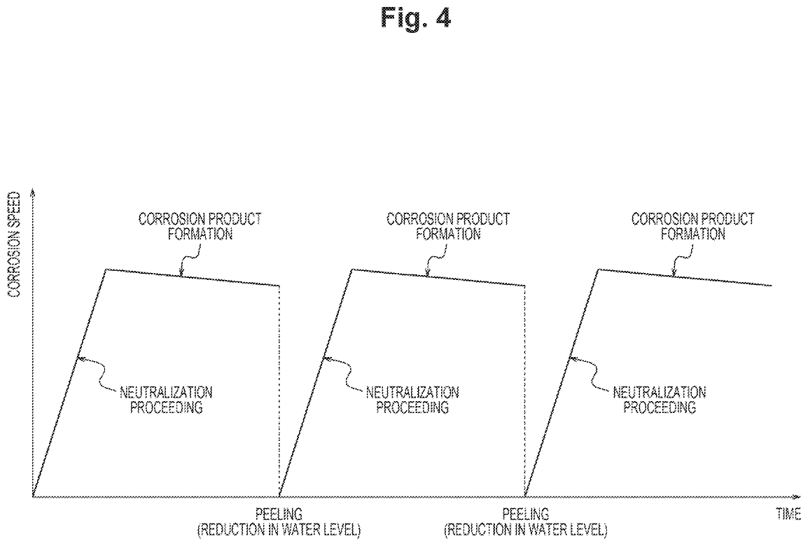

The corrosion state measured by the measurement control unit 251 includes, for example, a corrosion rate. For example, the measurement control unit 251 can measure the corrosion rate in the respective processes of the progress of neutralization of the calcium hydroxide aqueous solution 112, the formation of a corrosion product (for example, a corrosion coating) of the reinforcing bar member 113, and the decrease in the water level of the calcium hydroxide aqueous solution 112 corresponding to the peeling of the concrete as illustrated in FIG. 4 using a natural potential method, a method based on polarization resistance, or the like. In the example illustrated in FIG. 4, the corrosion rate increases as the neutralization of the calcium hydroxide aqueous solution 112 proceeds, and the corrosion rate slightly decreases as corrosion products are formed. As described above, when the relative position with respect to the interface of the reinforcing bar member 113 is controlled (for example, the water level decreases), the corrosion rate changes to 0. Thereafter, the corrosion rate increases again with the progress of neutralization of the calcium hydroxide aqueous solution 112, and the above-described change in the corrosion rate is repeated.

The analysis unit 252 can analyze an electrical signal indicating the characteristic of the reinforcing bar member 113 measured by the measurement control unit 251 by any method.

The measurement information storage unit 253 can store measurement information. The measurement information can be information indicating an electrical signal representing a characteristic of the reinforcing bar member 113 measured by the measurement control unit 251. In addition, the measurement information can be information indicating a result analyzed by the analysis unit 252.

### <Operation of Evaluation Device>

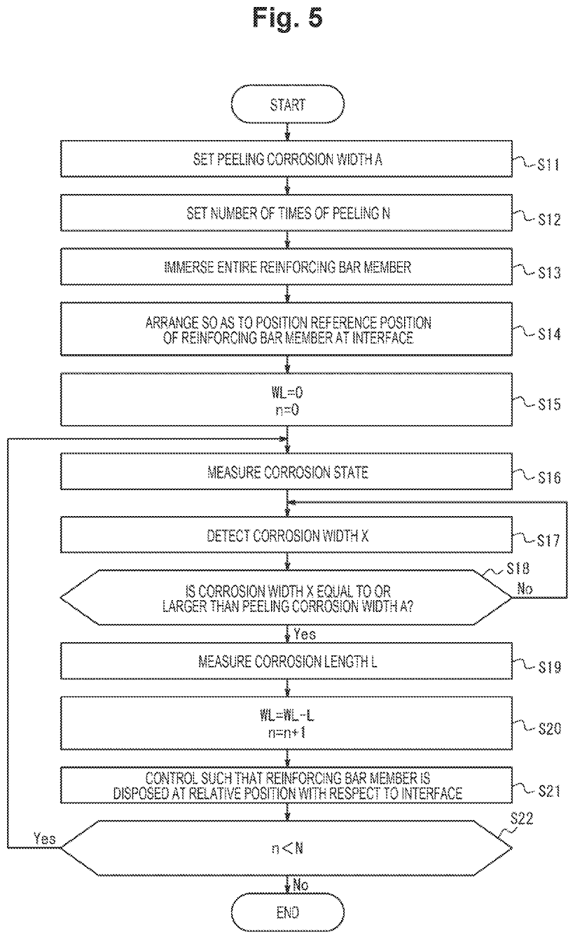

Here, an operation using the control device 2 according to the present embodiment will be described with reference to FIG. 5. FIG. 5 is a flowchart: illustrating an example of an operation using the control device 2 according to the present embodiment. The operation using the control device 2 described with reference to FIG. 5 includes an example of an estimation method of the control device 2 according to the present embodiment.

In step S11, the peeling corrosion width A is set. Specifically, the user may input the peeling corrosion width A to the control device 2, or another device may transmit the peeling corrosion width A to the control device 2. As a result, the input interface of the control device 2 receives the input of the peeling corrosion width A. Then, the setting value storage unit 22 stores the peeling corrosion width A input by the input interface.

In step S12, the number of times of peeling N is set. Specifically, the user may input the number of times of peeling N to the control device 2, or another device may transmit the number of times of peeling N to the control device 2. As a result, the input interface of the control device 2 receives the input of the number of times of peeling N. Then, the setting value storage unit 22 stores the number of times of peeling N when the input is received by the input interface.

In step S13, the entire reinforcing bar member 113 is immersed.

In step S14, the reinforcing bar member 113 is disposed such that the reference position is located at the interface. The reference position is a predetermined position in the reinforcing bar member 113, and is a position that is a boundary between a portion immersed in the calcium hydroxide aqueous solution 112 and a portion not immersed in the calcium hydroxide aqueous solution 112 in the initial state.

In step S15, the relative position determination unit 23 sets the water level WL to 0. The water level WL is the position of the interface of the calcium hydroxide aqueous solution 112 on the coordinate axis (the y axis in the example of FIGS. 3A to 3C) with the reference position as the origin (“0” in the example of FIGS. 3A to 3C) and the vertically upper side as the positive direction. The relative position determination unit 23 sets the natural number n to 0.

In step S16, the measurement unit 25 measures the corrosion state of the reinforcing bar member 113.

In step S17, the relative position determination unit 23 detects the corrosion amount of the corrosion product 113a generated in the reinforcing bar member 113.

Subsequently, from step S18 to step S20, the relative position determination unit 23 determines the relative position of the reinforcing bar member 113 with respect to the interface of the calcium hydroxide aqueous solution 112 on the basis of the corrosion amount.

Specifically, in step S18, the relative position determination unit 23 determines whether the corrosion width X is equal to or larger than the peeling corrosion width A.

When it is determined in step S18 that the corrosion width X is smaller than the peeling corrosion width A, the relative position determination unit 23 returns to step S17 and repeats the processing.

When it is determined in step S18 that the corrosion width X is equal to or larger than the peeling corrosion width A, in step S19, the relative position determination unit 23 measures corrosion length L that is a length of the corrosion product 113a generated in the portion of the reinforcing bar member 113 immersed in the calcium hydroxide aqueous solution 112.

In step S20, the relative position determination unit 23 determines the relative position of the reinforcing bar member 113 in the vertical direction with respect to the interface of the calcium hydroxide aqueous solution 112 on the basis of the corrosion length L. In the example illustrated in FIGS. 3A and 3B, the relative position determination unit 23 determines the relative position of the reinforcing bar member 113 with respect to the interface of the calcium hydroxide aqueous solution 112 as a position higher than the current relative position by the corrosion length L1. That is, the relative position determination unit 23 determines the interface to be a position lower than the current position of the interface by the corrosion length L1. Specifically, the relative position determination unit 23 determines that the water level WL=WL−D. The relative position determination unit 23 sets n=n+1.

In step S21, the relative position control unit 243 controls the reinforcing bar member 113 to be disposed at the relative position determined by the relative position determination unit 23 with respect to the interface.

In step S22, the relative position determination unit 23 determines whether n is smaller than the number of times of peeling N.

When it is determined in step S22 that n is smaller than the number of times of peeling N, the control device 2 returns to step S16 and repeats the processing.

When it is determined in step S22 that n is equal to or larger than the number of times of peeling N, the control device 2 ends the process.

As described above, the corrosion reproduction device 1 according to the present embodiment includes the container 111, the calcium hydroxide aqueous solution 112 accommodated in the container 111, and the reinforcing bar member 113 immersed in the calcium hydroxide aqueous solution 112. Accordingly, the corrosion reproduction device 1 can easily reproduce corrosion of reinforced concrete.

In the corrosion reproduction device 1 according to the present embodiment, the calcium hydroxide aqueous solution 112 is gelled. As a result, the corrosion reproduction device 1 can suppress the neutralization of the calcium hydroxide aqueous solution 112 from becoming non-uniform due to the convection of the calcium hydroxide aqueous solution 112. In addition, it is possible to suppress a change in the water level due to evaporation of the calcium hydroxide aqueous solution 112. Accordingly, the corrosion situation in the corrosion reproduction device 1 can be stably measured.

A control device 2 according to the present embodiment is a control device 2 that controls a corrosion reproduction device 1 including a container 111, a calcium hydroxide aqueous solution 112 accommodated in the container 111, and a reinforcing bar member 113 immersed in the calcium hydroxide aqueous solution 112, the control device 2 including: a corrosion amount detection unit 21 that detects a corrosion amount of a corrosion product 113a generated in the reinforcing bar member 113; a relative position determination unit 23 that determines a relative position of the reinforcing bar member 113 with respect to an interface of the calcium hydroxide aqueous solution 112 on the basis of the corrosion amount; and a relative position control unit 243 that performs control such that the reinforcing bar member 113 is disposed at the relative position with respect to the interface. As described above, the control device 2 can easily reproduce the repetition of the concrete peeling in the actual structure of reinforced concrete by controlling the relative position with respect to the interface of the reinforcing bar member 113 in the corrosion reproduction device 1.

In the control device 2 according to the present embodiment, the relative position determination unit 23 determines whether the corrosion width X that is the expansion amount of the corrosion product 113a in the direction parallel to the interface of the calcium hydroxide aqueous solution 112, is equal to or larger than the peeling corrosion width A, and when it is determined that the corrosion width X is equal to or larger than the peeling corrosion width A, the corrosion length L that is the length in the normal direction of the interface of the corrosion product 113a generated in the portion of the reinforcing bar member 113 immersed in the calcium hydroxide aqueous solution 112, is measured, and the relative position is determined on the basis of the corrosion length L. As a result, the control device 2 can set the boundary between the corroded portion and the non-corroded portion in the calcium hydroxide aqueous solution 112 of the reinforcing bar member 113 in the corrosion reproduction device 1 as the position of the interface of the calcium hydroxide aqueous solution 112. Accordingly, the control device 2 can accurately reproduce the concrete peeling.

The control device 2 according to the present embodiment further includes an environmental control unit 24 that controls the temperature or humidity of the environment including the calcium hydroxide aqueous solution 112 and the reinforcing bar member 113 or the concentration of the gas contained in the environment. As a result, the control device 2 can neutralize the calcium hydroxide aqueous solution 112 that reproduces in a pseudo manner the concrete at a higher speed than the speed at which the concrete is neutralized in the actual structure. In addition, the control device 2 can cause the reinforcing bar to corrode at a speed higher than the speed at which the reinforcing bar corrodes in the actual structure. Accordingly, the corrosion state of reinforced concrete can be evaluated more quickly than in a case of evaluating the corrosion state in the actual structure.

In addition, in the control device 2 according to the present embodiment, the measurement control unit 251 measures the corrosion state of the reinforcing bar member 113 by applying an electrical signal to the reference electrode 121, the counter electrode 122, and the reinforcing bar member 113 as a working electrode using a natural potential method, a polarization resistance method, or the like. As a result, it is not necessary to visually grasp the corrosion state of the portion buried in the concrete, and accordingly, it is not necessary to destroy the periphery of the portion.

### <Program>

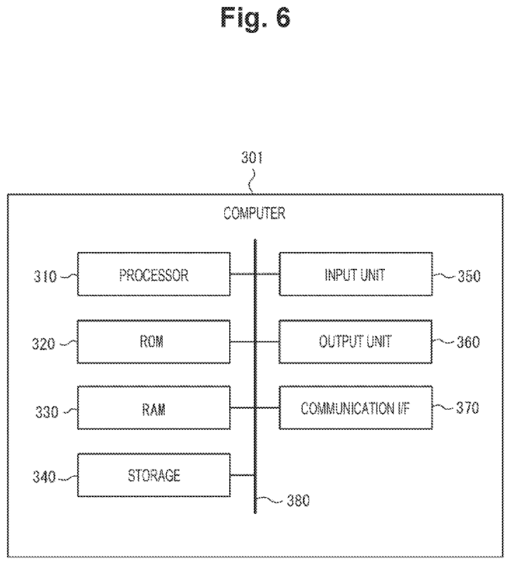

The control device 2 described above can be implemented by a computer 301. A program for functioning as the control device 2 described above may be provided. Furthermore, the program may be stored in a storage medium or may be provided via a network. FIG. 6 is a block diagram illustrating a schematic configuration of a computer 301 that functions as the control device 2. Here, the computer 301 may be a general-purpose computer, a dedicated computer, a workstation, a personal computer (PC), an electronic notepad, or the like. The program instructions may be program codes, code segments, and the like for executing a required task.

As illustrated in FIG. 6, the computer 301 includes a processor 310, a read only memory (ROM) 320, a random access memory (RAM) 330, a storage 340, an input unit 350, an output unit 360, and a communication interface (I/F) 370. The components are communicably connected with each other via a bus 380. Specifically, the processor 310 is a central processing unit (CPU), a micro processing unit (MPU), a graphics processing unit (GPU), a digital signal processor (DSP); a system on a chip (SoC), or the like, and may include a plurality of processors of the same or different types.

The processor 310 executes control on the components and various types of arithmetic processing. That is, the processor 310 reads the program from the ROM 320 or the storage 340 and executes the program by using the RAM 330 as a work area. The processor 310 performs control on the components and various types of arithmetic processing in accordance with the program stored in the ROM 320 or the storage 340. In the embodiments described above, the program according to the present disclosure is stored in the ROM 320 or the storage 340.

The program may be stored in a storage medium that can be read by the computer 301. By using such a storage medium, it is possible to install the program in the computer 301. Here, the storage medium in which the program is stored may be a non-transitory storage medium. The non-transitory storage medium is not particularly limited, but may be, for example, a CD-ROM, a DVD-ROM, a universal serial bus (USB) memory, or the like. The program may be downloaded from an external device via a network.

The ROM 320 stores various programs and various types of data. The RAM 330, as a work area, temporarily stores programs or data. The storage 340 includes a hard disk drive (HDD) or a solid state drive (SSD), and stores various programs including an operating system and various types of data.

The input unit 350 includes one or more input interfaces that receive a user's input operation and acquire information based on the user's operation. For example, the input unit 350 is a pointing device, a keyboard, a mouse, or the like, but is not limited to these.

The output unit 360 includes one or more output interfaces that output information. The output unit 360 is a display that outputs information as video images, or a speaker that outputs information as sound, for example, but is not limited to them. The output unit 360 also functions as the input unit 350 in a case where the output unit is a touch panel display.

The communication interface (I/F) 370 is an interface for communicating with an external device.

Regarding the above embodiments, the following supplementary notes are further disclosed.

### [Supplementary Note 1]

A corrosion reproduction device including
a container,
a calcium hydroxide aqueous solution accommodated in the container, and
a reinforcing bar member immersed in the calcium hydroxide aqueous solution.

### [Supplementary Note 2]

The corrosion reproduction device according to supplementary note 1, in which the calcium hydroxide aqueous solution is gelled.

### [Supplementary Note 3]

A control device that controls a corrosion reproduction device including a container, a calcium hydroxide aqueous solution accommodated in the container, and a reinforcing bar member immersed in the calcium hydroxide aqueous solution, the control device including
a controller,
in which the controller
detects a corrosion amount of a corrosion product generated in the reinforcing bar member,
determines a relative position of the reinforcing bar member with respect to an interface of the calcium hydroxide aqueous solution on the basis of the corrosion amount, and
performs control such that the reinforcing bar member is disposed at the relative position with respect to the interface.

1. A corrosion reproduction device comprising:
a container,
a calcium hydroxide aqueous solution accommodated in the container, and
a reinforcing bar member immersed in the calcium hydroxide aqueous solution.

2. The corrosion reproduction device according to claim 1, wherein the calcium hydroxide aqueous solution is gelled.

3. A control device for controlling the corrosion reproduction device according to claim 1,
the control device comprising:
a corrosion amount detection unit configured to detect a corrosion amount of a corrosion product generated in the reinforcing bar member;
a relative position determination unit configured to determine a relative position of the reinforcing bar member with respect to an interface of the calcium hydroxide aqueous solution based on the corrosion amount; and
a relative position control unit configured to perform control such that the reinforcing bar member is disposed at the relative position with respect to the interface.

4. The control device according to claim 3, wherein the relative position determination unit is configured to determine whether a corrosion width that is an expansion amount of the corrosion product in a direction parallel to the interface of the calcium hydroxide aqueous solution, is equal to or larger than a peeling corrosion width, and when the corrosion width is determined to be equal to or larger than the peeling corrosion width, a corrosion length that is a length in a normal direction of the interface of a corrosion product generated in a portion of the reinforcing bar member immersed in the calcium hydroxide aqueous solution, is measured, and the relative position is determined based on the corrosion length.

5. The control device according to claim 3, further comprising a temperature and humidity control unit configured to control a temperature or humidity of an environment in which the calcium hydroxide aqueous solution and the reinforcing bar member are provided.

6. An evaluation system comprising:
a corrosion reproduction device including:
a container,
a calcium hydroxide aqueous solution accommodated in the container, and
a reinforcing bar member immersed in the calcium hydroxide aqueous solution; and
a control device including:
a corrosion amount detection unit configured to detect a corrosion amount of a corrosion product generated in the reinforcing bar member,
a relative position determination unit configured to determine a relative position of the reinforcing bar member with respect to an interface of the calcium hydroxide aqueous solution based on the corrosion amount, and
a relative position control unit configured to perform control such that the reinforcing bar member is disposed at the relative position with respect to the interface.

7. A method comprising:
detecting, by a control device, a corrosion amount of a corrosion product generated in a reinforcing bar member in a corrosion reproduction device, the corrosion reproduction device including a container, a calcium hydroxide aqueous solution accommodated in the container, and the reinforcing bar member immersed in the calcium hydroxide aqueous solution;
determining, by the control device, a relative position of the reinforcing bar member with respect to an interface of the calcium hydroxide aqueous solution based on the corrosion amount; and
performing, by the control device, control such that the reinforcing bar member is disposed at the relative position with respect to the interface.

8. A computer-readable memory device storing computer-executable program instructions that, when executed by a processor, cause a computer to function as the control device according to claim 3.

9. A computer-readable memory device storing computer-executable program instructions that, when executed by a processor, cause a computer to function as the control device according to claim 4.

10. The control device according to claim 4, further comprising a temperature and humidity control unit configured to control a temperature or humidity of an environment in which the calcium hydroxide aqueous solution and the reinforcing bar member are provided.

11. The control device according to claim 3, further comprising a gas control unit configured to control a concentration of a gas contained in the environment.

12. The control device according to claim 4, further comprising a gas control unit configured to control a concentration of a gas contained in the environment.

13. The evaluation system according to claim 6, wherein the relative position determination unit is configured to determine whether a corrosion width that is an expansion amount of the corrosion product in a direction parallel to the interface of the calcium hydroxide aqueous solution, is equal to or larger than a peeling corrosion width, and when the corrosion width is determined to be equal to or larger than the peeling corrosion width, a corrosion length that is a length in a normal direction of the interface of a corrosion product generated in a portion of the reinforcing bar member immersed in the calcium hydroxide aqueous solution, is measured, and the relative position is determined based on the corrosion length.

14. The evaluation system according to claim 6, the control device further including a temperature and humidity control unit configured to control a temperature or humidity of an environment in which the calcium hydroxide aqueous solution and the reinforcing bar member are provided.

15. The evaluation system according to claim 6, the control device further including a gas control unit configured to control a concentration of a gas contained in the environment.

16. The method according to claim 7, further comprising:
determining whether a corrosion width that is an expansion amount of the corrosion product in a direction parallel to the interface of the calcium hydroxide aqueous solution, is equal to or larger than a peeling corrosion width;
when the corrosion width is determined to be equal to or larger than the peeling corrosion width, measuring a corrosion length that is a length in a normal direction of the interface of a corrosion product generated in a portion of the reinforcing bar member immersed in the calcium hydroxide aqueous solution; and
determining the relative position based on the corrosion length.

17. The method according to claim 7, further comprising:
controlling, by a temperature and humidity control unit, a temperature or humidity of an environment in which the calcium hydroxide aqueous solution and the reinforcing bar member are provided.

18. The method according to claim 7, further comprising:
controlling, by a gas control unit, a concentration of a gas contained in the environment.
